(19) United States
(12) Patent Application Publication
(10) Pub. No.: US 2024/0082448 A1
(43) Pub. Date: Mar. 14, 2024

(54) Tabletop Scent Dispensing Device

(71) Applicant: Pura Scents, Inc., Pleasant Grove, UT (US)

(72) Inventors: Rachel Beckstead, Lehi, UT (US); Celeste Chubak, West Valley City, UT (US); Hannah Trimble, Salt Lake City, UT (US); Juan Carlos Altuna, South Jordan, UT (US); David Frietag, Pleasant Grove, UT (US); John Adam Purin, Ogden, UT (US); Kenneth Frei, Lehi, UT (US)

(21) Appl. No.: 18/466,505

(22) Filed: Sep. 13, 2023

Related U.S. Application Data
(60) Provisional application No. 63/514,960, filed on Jul. 21, 2023.
(60) Provisional application No. 63/375,670, filed on Sep. 14, 2022.

(51) Int. Cl. A61L 9/03

(52) U.S. Cl. CPC A61L 9/032; A61L 2209/11

(57) ABSTRACT

A scent dispensing device may comprise a scent vial holder configured to hold a scent vial, a top having a plurality of openings, the plurality of openings including a scent opening and an airflow opening, a heater configured to apply heat to the scent vial, and a fan configured to cause a flow of air to pass through the airflow opening creating a difference in air pressure between the airflow opening and the scent opening such that scent from the scent vial is drawn through the scent opening and into the flow of air.

### CROSS-REFERENCE TO RELATED APPLICATIONS

The present application claims the benefit, under 35 U.S.C. § 119, of U.S. Provisional Patent Application No. 63/375,670, filed Sep. 14, 2022 and titled “Tabletop Fragrance Dispensing Device” and U.S. Provisional Patent Application No. 63/514,960, filed Jul. 21, 2023 and titled “Tabletop Fragrance Dispensing Device”, which are incorporated herein by reference in their entirety.

### BACKGROUND

The present disclosure relates to scent dispensation.

Existing solutions for dispensing scents within a room include liquid scented oil diffusers, wax or cork disks infused with scented oils, or nebulizers. The scented oil can be dispersed throughout the room by heating elements and/or fans. However, such diffusers typically require connection to a power source (e.g., a wall outlet) and cannot be effectively located in a room due to the need to be proximate to a power source. Furthermore, the scent being diffused cannot easily be changed in existing solutions.

### SUMMARY

In some aspects, the techniques described herein relate to a scent dispensing device, including: a scent vial holder configured to hold a scent vial; a top having a first plurality of openings, the first plurality of openings including a scent opening and an airflow opening; a heater configured to apply heat to the scent vial; and a fan configured to cause a flow of air to pass through the airflow opening creating a difference in air pressure between the airflow opening and the scent opening such that scent from the scent vial is drawn through the scent opening and into the flow of air.

In some aspects, the techniques described herein relate to a scent dispensing device, further including: a controller configured to control the heater and the fan to achieve a selected scent diffusion level based on user input.

In some aspects, the techniques described herein relate to a scent dispensing device, further including a wick cover having a second plurality of openings, the wick cover configured to at least partially close the scent opening.

In some aspects, the techniques described herein relate to a scent dispensing device, further including an actuator coupled with the wick cover, the actuator configured to adjust a position of the wick cover.

In some aspects, the techniques described herein relate to a scent dispensing device, wherein the controller is further configured to control the actuator such that the second plurality of openings of the wick cover are positioned to further achieve the selected scent diffusion level.

In some aspects, the techniques described herein relate to a scent dispensing device, further including a tag reader configured to read a tag coupled with the scent vial.

In some aspects, the techniques described herein relate to a scent dispensing device, further including: a base having a third plurality of openings; and an air channel positioned between the fan and the airflow opening, wherein the fan is configured to draw air through the third plurality of openings and through the air channel such that scent molecules are not drawn over internal components of the scent dispensing device.

In some aspects, the techniques described herein relate to a scent dispensing device, including: a scent vial holder configured to hold a plurality of scent vials; a tag reader configured to read tags coupled with the plurality of scent vials; a top having a first plurality of openings, the first plurality of openings including a plurality of scent openings and an airflow opening; a plurality of heating elements configured to selectively apply heat to the plurality of scent vials; a fan configured to cause a flow of air to pass through the airflow opening creating a difference in air pressure between the airflow opening and the plurality of scent openings such that one or more scents from one or more of the plurality of scent vials is drawn through the plurality of scent openings and into the flow of air; and a controller configured to control the plurality of heating elements and the fan to achieve a selected scent diffusion level of the one or more scents.

In some aspects, the techniques described herein relate to a scent dispensing device, further including a wick cover having a second plurality of openings, the wick cover configured to at least partially close one or more of the plurality of scent openings.

In some aspects, the techniques described herein relate to a scent dispensing device, further including an actuator coupled with the wick cover, the actuator configured to adjust a position of the wick cover.

In some aspects, the techniques described herein relate to a scent dispensing device, wherein the controller is further configured to control the actuator such that the second plurality of openings of the wick cover are positioned to further achieve the selected scent diffusion level.

In some aspects, the techniques described herein relate to a scent dispensing device, wherein the controller is further configured to identify a scent contained in each of the plurality of scent vials based on an output from the tag reader.

In some aspects, the techniques described herein relate to a scent dispensing device, wherein the controller is further configured to: selectively apply heat to a first scent vial of the plurality of scent vials; and control the actuator such that the wick cover is not covering a first scent opening corresponding to the first scent vial and is covering remaining scent openings of the plurality of scent openings to achieve the selected scent diffusion level.

In some aspects, the techniques described herein relate to a scent dispensing device, further including: a base having a third plurality of openings; and an air channel positioned between the fan and the airflow opening, wherein the fan is configured to draw air through the third plurality of openings and through the air channel such that scent molecules are not drawn over internal components of the scent dispensing device.

In some aspects, the techniques described herein relate to a method including: receiving, by a controller of a scent dispensing device, user input; determining, by the controller of the scent dispensing device based on the user input, a scent diffusion level; controlling, by the controller of the scent dispensing device, a heating element of the scent dispensing device to achieve the scent diffusion level; and controlling, by the controller of the scent dispensing device, a fan of the scent dispensing device to achieve the scent diffusion level.

In some aspects, the techniques described herein relate to a method, wherein determining the scent diffusion level includes identifying a plurality of scent parameters and determining the scent diffusion level based on the plurality of scent parameters.

In some aspects, the techniques described herein relate to a method, wherein the plurality of scent parameters include one or more of: a scent selection, a run time, a scent mixture, a room size, a time of day, an ambient temperature, and a scent level preference.

In some aspects, the techniques described herein relate to a method, wherein controlling the fan of the scent dispensing device further includes operating the fan in a pulse mode such that bursts of scent are dispersed to achieve the scent diffusion level.

In some aspects, the techniques described herein relate to a method, further including: controlling, by the controller of the scent dispensing device, an actuator coupled with a wick cover of the scent dispensing device to cover a scent vial wick when the scent dispensing device is not in use.

In some aspects, the techniques described herein relate to a method, further including: identifying, by the controller of the scent dispensing device, a scent vial based on input from a tag reader configured to read a tag coupled with the scent vial held in the scent dispensing device.

However, this list of features and advantages is not all-inclusive and many additional features and advantages are within the scope of the present disclosure. Moreover, it should be noted that the language used in the present disclosure has been principally selected for readability and instructional purposes, and not to limit the scope of the subject matter disclosed herein.

### BRIEF DESCRIPTION OF THE DRAWINGS

FIG. 1 is a block diagram illustrating an example system for scent dispensation.

FIG. 2A illustrates an example of the scent dispensing device.

FIG. 2B illustrates an example of the scent dispensing device with the lid removed.

FIG. 2C illustrates an example of the scent dispensing device with the carousel raised.

FIG. 2D illustrates an example lid of the scent dispensing device.

FIG. 2E illustrates example internal components of a lid of the scent dispensing device.

FIG. 3 depicts an example of the scent dispensing device coupled with a wireless charging base.

FIG. 4A is a top perspective view of an example scent dispensing device.

FIG. 4B is a bottom perspective view of the example scent dispensing device.

FIG. 4C is a front view of the example scent dispensing device.

FIG. 4D is a left side view of the example scent dispensing device.

FIG. 4E is a bottom view of the example scent dispensing device.

FIG. 4F is a right side view of the example scent dispensing device.

FIG. 4G is a top view of the example dispensing device.

FIG. 4H is a rear view of the example dispensing device.

FIG. 5 illustrates example internal components of a scent dispensing device.

FIG. 6A is an example embodiment of another scent dispensing device.

FIG. 6B is another example of the scent dispensing device introduced in FIG. 6A.

FIG. 6C is another example of the scent dispensing device shown in FIG. 6A.

FIG. 6D is another example of the scent dispensing device shown in FIG. 6A.

FIG. 7A is a top perspective view of another example scent dispensing device.

FIG. 7B is a bottom perspective view of the example scent dispensing device.

FIG. 7C is a front view of the example scent dispensing device.

FIG. 7D is a left side view of the example scent dispensing device.

FIG. 7E is a bottom view of the example scent dispensing device.

FIG. 7F is a right side view of the example scent dispensing device.

FIG. 7G is a top view of the example dispensing device.

FIG. 7H is a rear view of the example dispensing device.

FIG. 8 is a flowchart of an example method for dispensing a scent using a scent dispensing device.

### DETAILED DESCRIPTION

The technology described in this disclosure relates to dispensing scent within a space using a scent dispensing device. As an example, the technology allows a scent dispensing device to be positioned on any surface in a space and dispense a chosen scent efficiently in the space without the need to be connected to a power source.

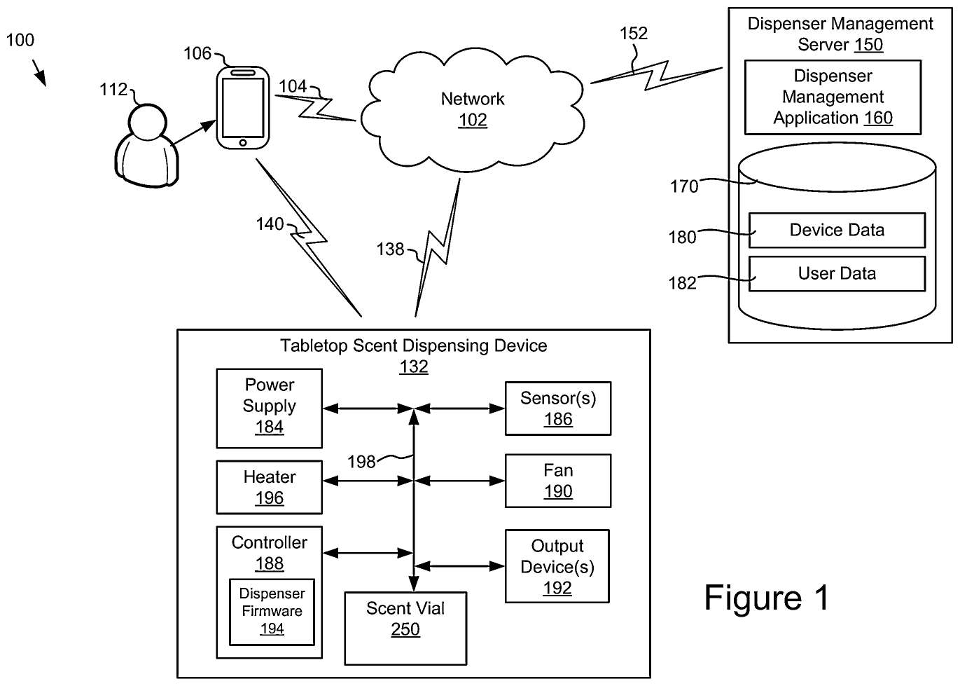

FIG. 1 is a block diagram illustrating an example system 100 for scent dispensation. The system 100 may include a scent dispensing device 132 positioned on a surface (e.g., a tabletop, counter, shelf, etc.) within a space. In a typical implementation, a scent dispensing device 132 is positioned within a space. However, in large spaces, multiple scent dispensing devices may be positioned throughout the space for effective scent dispensing. In some implementations, the scent dispensing device 132 is configured to sit on any surface (e.g., a tabletop, counter, shelf, etc.) and is portable and may be moved as needed while operating on battery power. In other implementations, the scent dispensing device 132 may include a wireless charging base which, when the scent dispensing device 132 rests on the base, charges the battery of the scent dispensing device 132.

As shown, the example system 100 further includes a user device 106 and a dispenser management server 150, which are electronically communicatively coupled via a network 102 for interaction with one another and the scent dispensing device 132, etc., using standard networking protocols, as reflected by signal line 104, 138, and 152. In some implementations, the scent dispensing device 132 may instead by communicatively coupled to the user device 106, such as by Bluetooth, beacons, or other networking protocols (as reflected by signal line 140) in order to allow the scent dispensing device 132 to connect with a paired user device 106 when in proximity rather than through the network 102.

The dispenser management application 160 operable by the dispenser management server 150 can receive operational data from the scent dispensing device 132 in association with the user device 106 and/or user 112 with which they are associated. The dispenser management application can receive management requests for a scent dispensing device 132, such as a diffusion level, scent choice, or various data that can be analyzed over time to improve the scent dispensing experience for the user 112.

The dispenser management server 150 includes a data store 170 storing various types of data used by the dispenser management application 160. Example data types include device data 180 and user data 182. The device data 180 may include a device model, a scent vial type, usage statistics, scent diffusion time, temperature variations, etc. The user data 182 may include entries for each user 112 of the system 100. A given entry may include a unique identifier for the user, a unique identifier for the user device 106, contact information for the user (e.g., address, phone number, electronic address (e.g., email)), payment information, scent subscription information specifying which reoccurring scent vials should be shipped to the user, etc.

An example scent dispensing device 132 is depicted as including a power supply 184, one or more of a sensor 186, a controller 188, an output device 192, dispenser firmware 194, a fan 190, a heater 196, and a scent vial 250. The components 184, 186, 188, 190, 192, 196, and 250 are communicatively coupled via a communications bus 198. The controller 188 may include a non-transitory memory device, or may be coupled to a non-transitory memory device also coupled for communication via the bus 198. The non-transitory memory device may store software and/or firmware that specially configures the controller, such as the dispenser firmware 194. The power supply 184 may be any AC and/or DC power supply for powering the scent dispensing device 132. In some implementations, the power supply 184 may be battery powered and may be configured to charge when plugged into an AC and/or DC power supply or positioned on a wireless charging base. The controller 188 may be a microchip that controls the constituent electronics (e.g., sensor 186, output device 192, fan 190, heater 196, etc.) of the scent dispensing device 132.

The one or more sensors 186 may include one or more temperature sensors for detecting the ambient temperature adjacent to the scent dispensing device 132 and can use those readings to determine efficient diffusion of the scent.

In some implementations, the one or more sensors 186 may include a vial sensor for sensing when to replace a scent vial 250 installed in the scent dispensing device 132, optical, near field communication, or other sensors or electronics for detecting an identity of a scent vial 250 installed in the scent dispensing device 132, ambient light sensor to detect a light level in a surrounding environment, and/or a motion sensor to detect motion in the surrounding environment, etc. In some embodiments, the sensors 186 may include a separate temperature sensor for the scent vial 250 (e.g., to measure temperatures at the scent vial 250, which can be used to adjust a speed and/or frequency of the fan 190).

The sensors 186 may include a transceiver having a wireless interface configured to communicate with the devices coupled to the network 102, such as the dispenser management server 150, and/or other components of the network 102 using standard communication protocols, such as Internet protocols. Further, the transceiver may be configured to wirelessly transmit data via a network to connect to other devices, such as the user device 106. By way of further example, the transceiver may transmit data to the user device 106 to which it is linked using a protocol compliant with IEEE 802.15, such as Zigbee®, Z-Wave®, Bluetooth®, or another suitable standard. Further embodiments are also possible and contemplated. In some embodiments, the transceiver may be embedded in the controller 188 or may be a component distinct from the controller and coupled to the controller 188 via the bus 198.

The output device 192 may include light sources and/or audio reproduction devices, although further suitable output devices are also contemplated and applicable. In some implementations, the light sources and/or audio reproduction devices may be controlled to produce output consistent with a scent being emitted by the scent dispensing device 132 (e.g., a low, soothing light and music may be output in conjunction with a relaxing scent being emitted), or to communicate various alerts, such as low power, low scent vial levels, etc.

The fan 190 may include a motor that has one or more fan blades that force air through the scent dispensing device 132 when the motor is operating. The fan 190 may operate at various speeds based on how quickly or slowly the motor runs. The fan 190 may be configured to nest within a housing of the scent dispensing device 132 and cause airflow to move through the device and across or near a scent vial 250 for scent diffusion. In some implementations, the fan 190 may create a change in the air pressure. For example, the fan 190 may cause the air to flow over the heater opening. In another example, the fan 190 may cause the air to flow next to an opening (such as an airflow opening or scent opening) and create a change in air pressure that draws the scent into the airflow stream. The air within the housing may be at a higher pressure than the air flowing through the airflow stream generated by the fan 190 in order to create a low pressure flow to draw out the air within the housing, or portions of the housing, such as the scent opening or heater opening, etc. In some implementations, the fan 190 may be able to turn on and off as signaled by the controller 188 which results in substantially immediate scent diffusion. In some implementations the fan 190 and the motor speed may be associated with scent settings and based on the level of the motor operating the fan 190 different scent diffusions can be released to fill different areas over different times based on the scent settings.

The scent vial 250 may be removable and contain a liquid scenting agent that diffuses a scent into the nearby air. The scent vial 250 may be replaceable when the scent is diminished and a new scent vial 250 can be inserted into the scent dispensing device 132. The scent vial 250 may have various scent profiles and information about the scent vial 250 may be stored in the dispenser management application 160 to store the various scent profiles, age of the scent vial 250, duration of use of the scent vial 250, exposed temperature of the scent vial 250, etc.

The user device 106 may be a computing device having data processing and communication capabilities. In some embodiments, a user device 106 may include a processor (e.g., virtual, physical, etc.), a memory, a power source, a network interface, and/or other software and/or hardware components, such as a display, graphics processor, wireless transceivers, keyboard, camera, sensors, firmware, operating systems, drivers, various physical connection interfaces (e.g., USB, HDMI, etc.).

The user device 106 may couple to and communicate with one another and the other entities of the system 100 via the network 102 using a wireless and/or wired connection. Examples of a user device 106 may include, but are not limited to, mobile phones (e.g., feature phones, smart phones, etc.), tablets, smartwatches or other smart wearables, laptops, desktops, netbooks, server appliances, servers, virtual machines, TVs, set-top boxes, media streaming devices, portable media players, navigation devices, personal digital assistants, car access panels, etc. In addition, while a single user device 106 is depicted in FIG. 1, it should be understood that any number of user devices 106 may be included.

As shown, the user device 106 may include a scent application, which allows the user to set scent dispensing device 132 settings, turn the scent dispensing device 132 on and off, purchase a scent vial 250 for the scent dispensing device 132, set up a scent dispensing device 132, register an account, view analytics reflecting the user's historical use of the scent dispensing device 132, enable user profiles to use and setup scent profiles for the scent dispensing device 132, set a profile hierarchy (e.g., set which user profile(s) is/are the dominant user profile), set motor speeds for the fan 190, manage various scent settings, etc.

The dispenser management server 150 may include one or more computing devices having data processing, storing, and communication capabilities. For example, the dispenser management server 150 may include one or more hardware servers, virtual servers, server arrays, storage devices and/or systems, etc., and/or may be centralized or distributed/cloud-based. In some embodiments, the dispenser management server 150 may include one or more virtual servers, which operate in a host server environment and access the physical hardware of the host server including, for example, a processor, memory, storage, network interfaces, etc., via an abstraction layer (e.g., a virtual machine manager).

While not depicted, the dispenser management server 150 may include a (physical, virtual, etc.) processor, a non-transitory memory, a network interface, and a data store 170, which may be communicatively coupled by a communications bus. Similarly, the user device 106 may include a physical processor, a non-transitory memory, a network interface, a display, an input device, a sensor, and a capture device. It should be understood that the server and the client device may take other forms and include additional or fewer components without departing from the scope of the present disclosure.

Software operating on the dispenser management server 150 (e.g., the dispenser management application 160, an operating system, device drivers, etc.) may cooperate and communicate via a software communication mechanism implemented in association with a server bus. The software communication mechanism can include and/or facilitate, for example, inter-process communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication could be secure (e.g., SSH, HTTPS, etc.).

As shown, the dispenser management server 150 may include a dispenser management application 160 embodying a remotely accessible scent service. The dispenser management application 160 may send data to and receive data from the other entities of the system including the controllers 188, the user device 106, etc. The dispenser management application 160 may be configured to store and retrieve data from one or more information sources, such as the data store 170. In addition, while one dispenser management server 150 is depicted in FIG. 1, it should be understood that one or more dispenser management servers 150 may be included.

In some embodiments, the dispenser firmware 194, the scent application, the dispenser management application 160, etc., may require users to be registered to access the acts and/or functionality provided by them. For example, to access various acts and/or functionality provided by the scent application, dispenser management application 160, and/or scent dispensing device 132, these components may require a user to authenticate his/her identity (e.g., by confirming a valid electronic address). In some instances, these entities may interact with a federated identity server (not shown) to register/authenticate users. Once registered, these entities may require a user seeking access to authenticate by inputting credentials in an associated user interface.

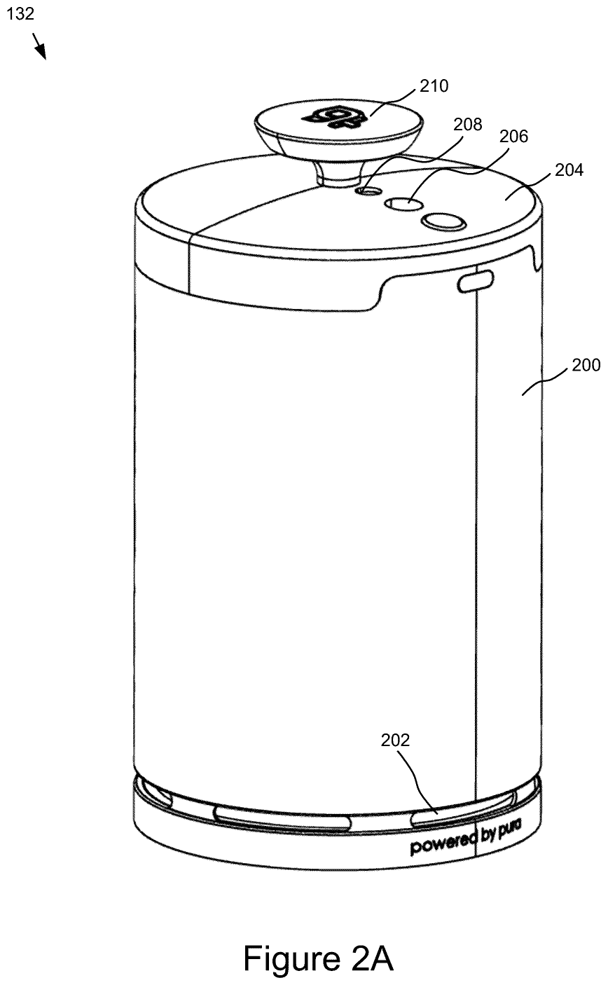

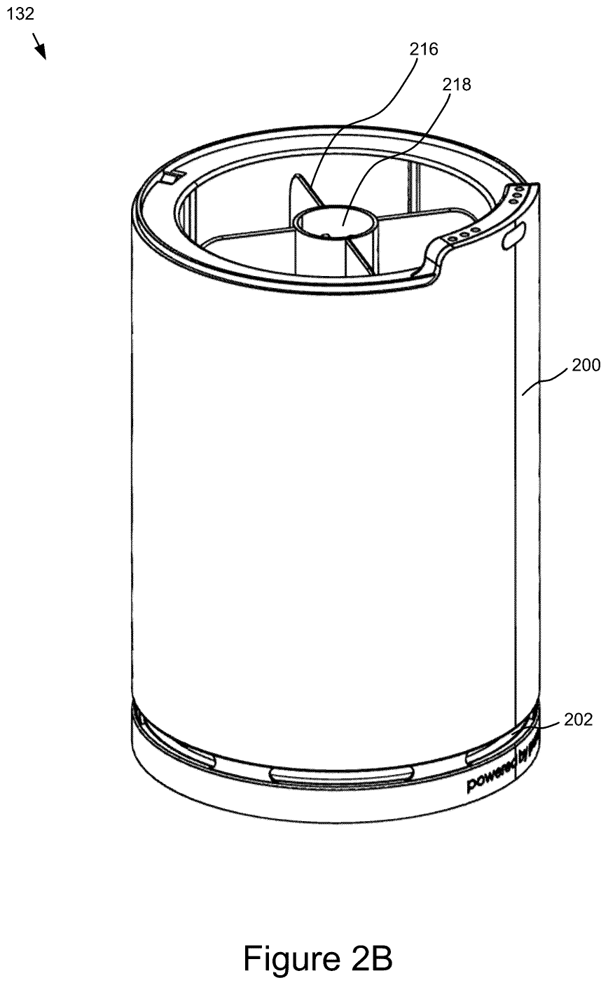

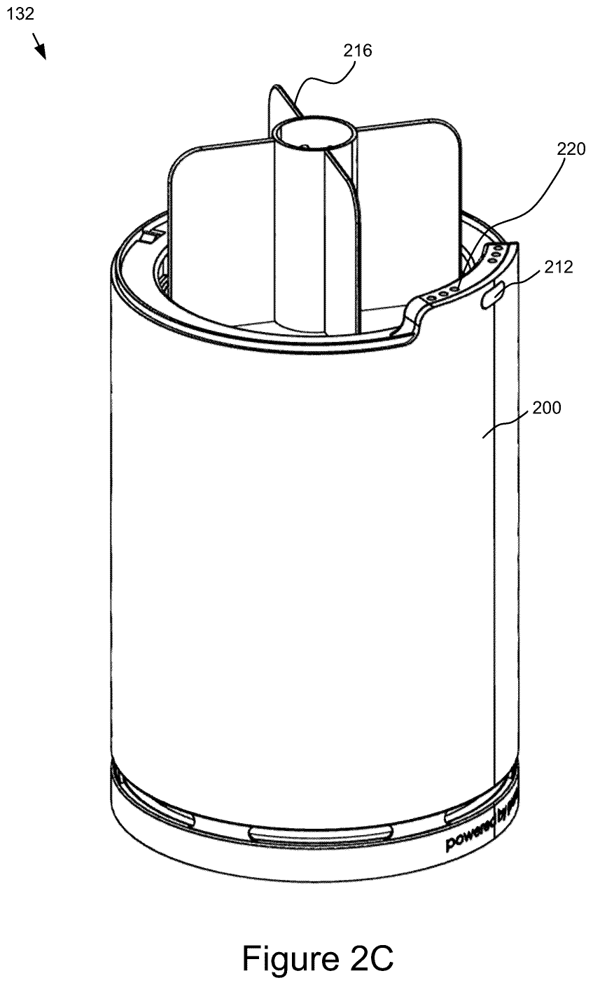

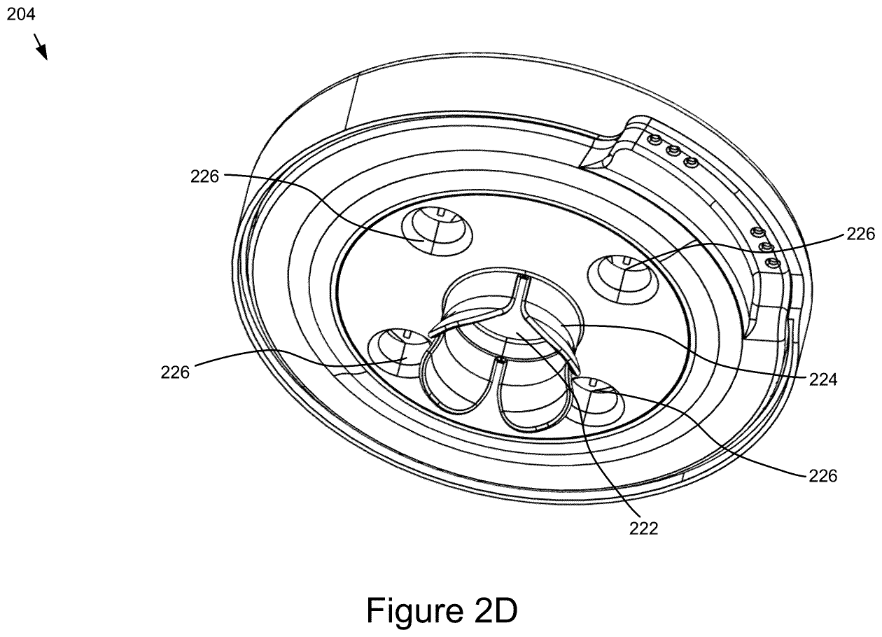

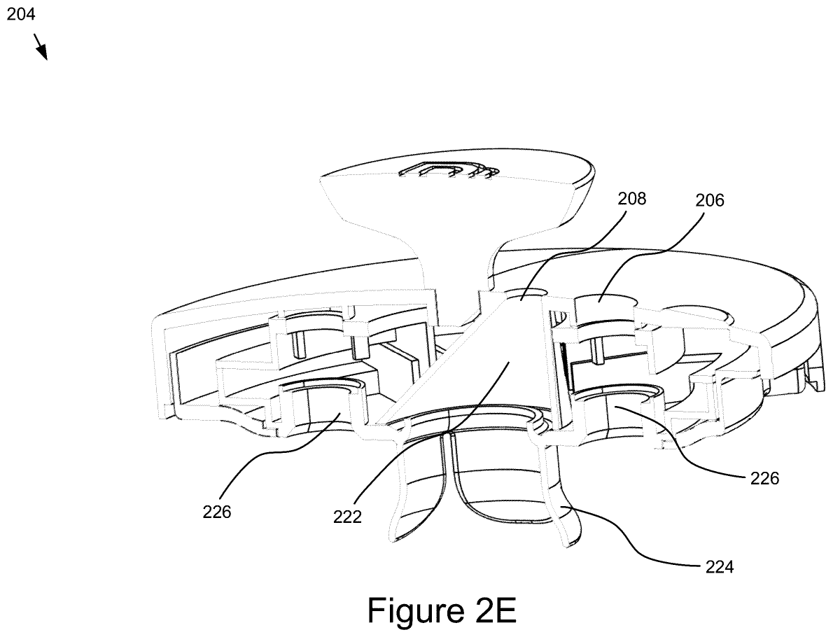

FIGS. 2A-2E are various views of an example scent dispensing device 132. As shown in FIG. 2A, the scent dispensing device 132 includes an outer housing 200 that houses the components of the scent dispensing device 132 (e.g., scent vial 250, fan 190, power supply 184, sensor 186, etc.). In some implementations, as shown, the scent dispensing device 132 may be cylindrical in shape with flat bottom surface, however, other shapes and configurations are also contemplated. The outer housing 200 includes openings 202 near the base of the scent dispensing device 132 to allow for air to be drawn in by the fan 190. The scent dispensing device 132 includes a top 204 that sits on the device and is removable to access the interior components of the scent dispensing device 132. The top 204 includes a scent opening 206 to allow for the scent from a scent vial 250 to exit the device and an opening 208 for air drawn through the device by fan 190 to exit the device and disperse the scent. The top 204 also includes a knob 210 which provides a convenient location for a user 112 to grasp and remove the top 204. In some embodiments, the outer housing 200, top 204, and/or knob 210 are interchangeable such that the shape, color, and/or design of the scent dispensing device 132 can be customized.

FIG. 2B depicts the scent dispensing device 132 with the top 204 removed. The interior of the scent dispensing device 132 includes a scent vial holder 216 which is configured to hold one or more scent vials (not shown). As shown in the example of FIG. 2B, the scent vial holder 216 is carousel shaped and can be rotated based on the scent selected by a user, as described further herein. If the example of FIG. 2B, the scent vial holder 216 is divided into four compartments, each of which can hold a scent vial 250. In other embodiments, the scent vial holder 216, may be divided into any number of compartments. The scent vial holder 216 includes a central airflow channel 218 which directs the air pulled in from the openings 202 through the opening 208 in the top 204.

When the top 204 is removed (e.g., identified by the scent dispensing device 132 when contacts 220 no longer detect the top) a motor in the scent dispensing device 132 raises the scent vial holder 216 to present the compartments to the user for easy access as depicted in the example of FIG. 2C. In some embodiments, the scent vial holder 216 may twist as it is raised to create a swirling presentation. The scent vial holder 216 may be lowered back down into the scent dispensing device 132 by pressing the button 212. In some embodiments, the button 212 includes a visual element, such as a light, to indicate that the button 212 is to be pressed to lower the carousel.

FIG. 2D shows an example of the top 204 of scent dispensing device 132. The top 204 may include airflow opening 222 which couples with the airflow channel 218 with the opening 208 and directs air across the scent opening 206. The opening 222 is defined by petal-like structures 224 that fit over the walls of the scent vial holder 216 and substantially limit air from the airflow channel 218 from flowing directly across a scent vial. As depicted in the example, the top 204 includes four internal scent openings 226 which correspond with the four compartments of scent vial holder 216 and corresponding scent vials contained therein. In some embodiments, the user may select (e.g., using a scent application on user device 106) the scent vial which is to be aligned with the scent opening 206. In response to the selection, the motor may rotate the scent vial holder 216 so that the selected scent is aligned with the scent opening 206. A cross-section of top 204 is shown in the example of FIG. 2E. As can be seen in FIG. 2E, the opening 222 funnels air to opening 208 and on of the four internal scent openings 226 (depending on which scent is selected by the user) is aligned with scent opening 206 in the top 204.

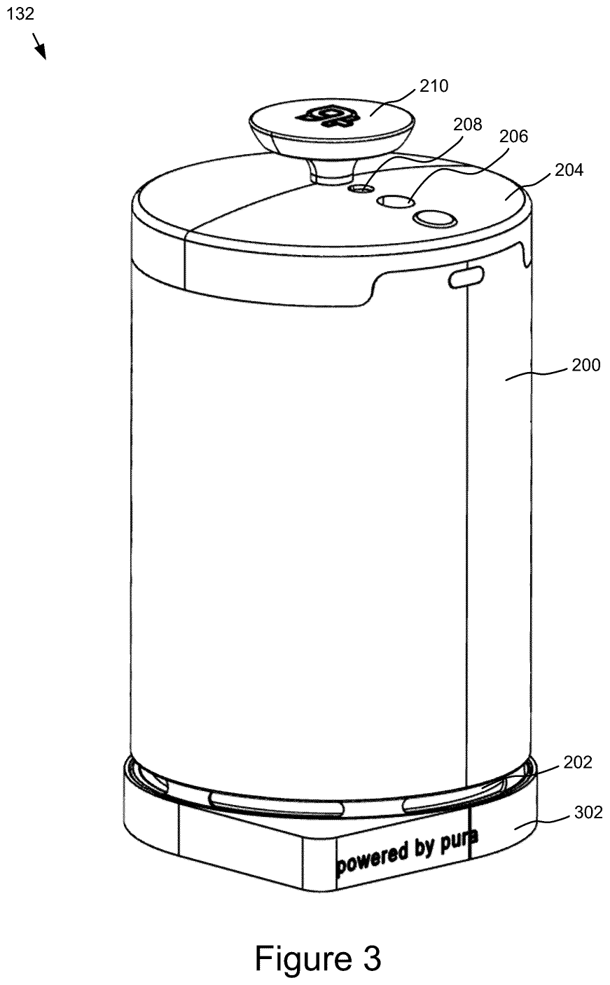

FIG. 3 depicts an example of the scent dispensing device 132 coupled with a wireless charging base 302.

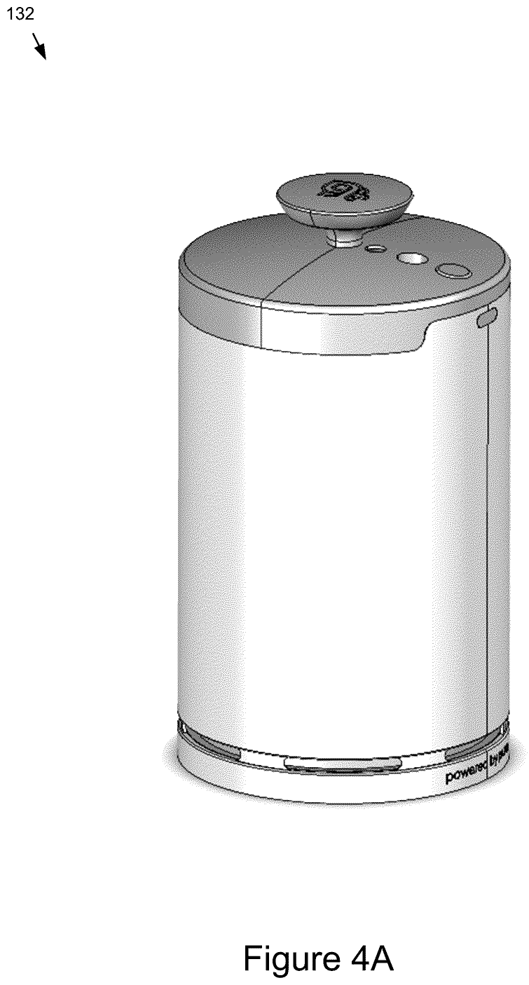

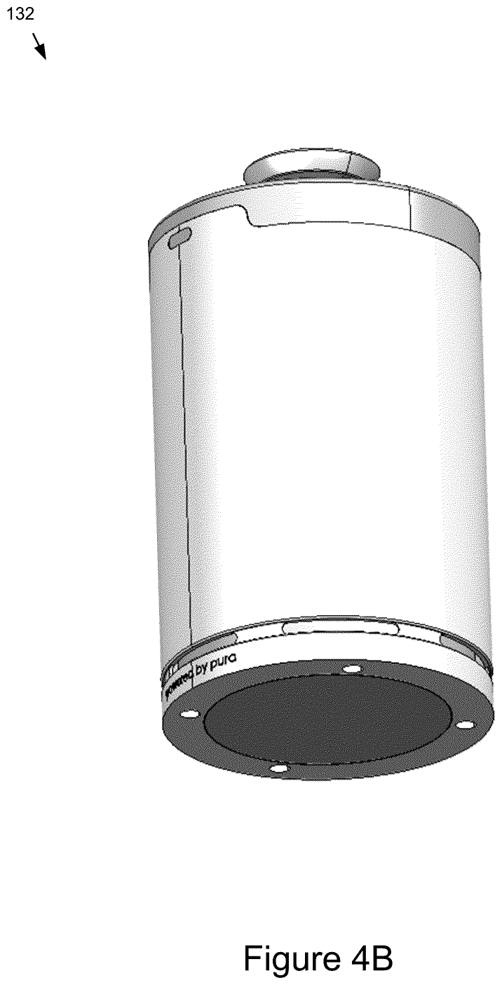

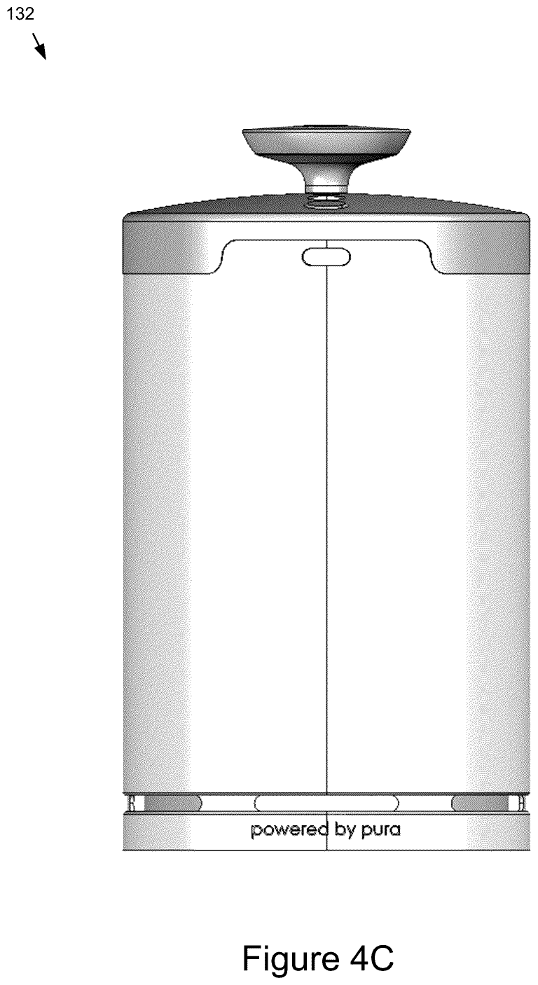

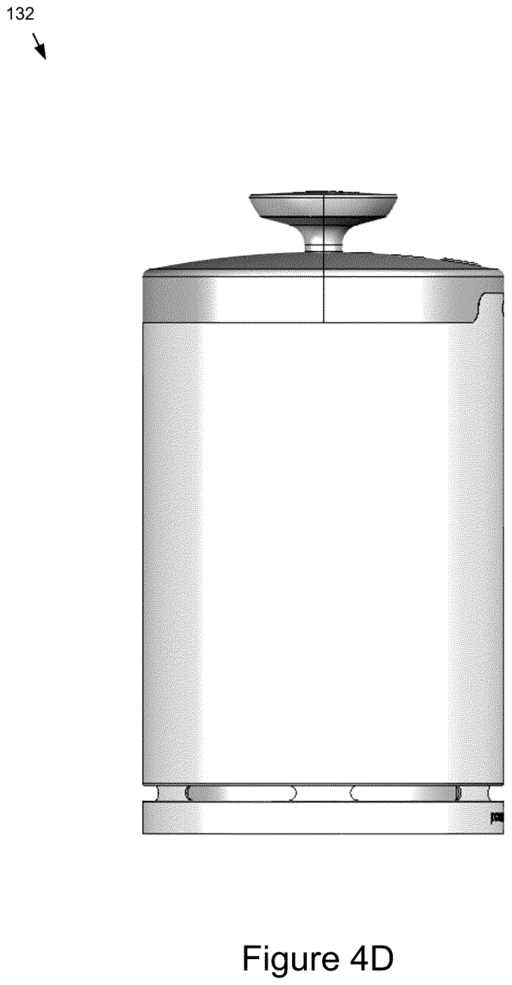

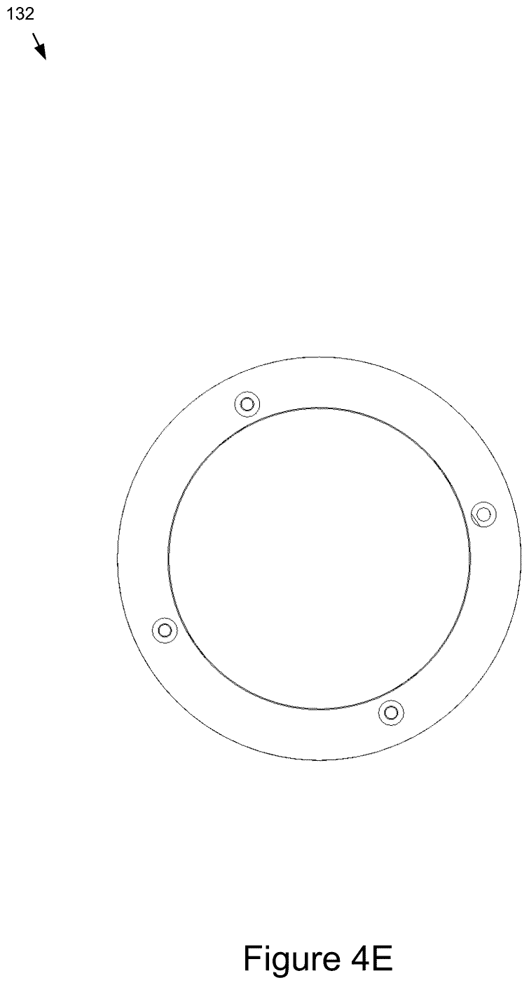

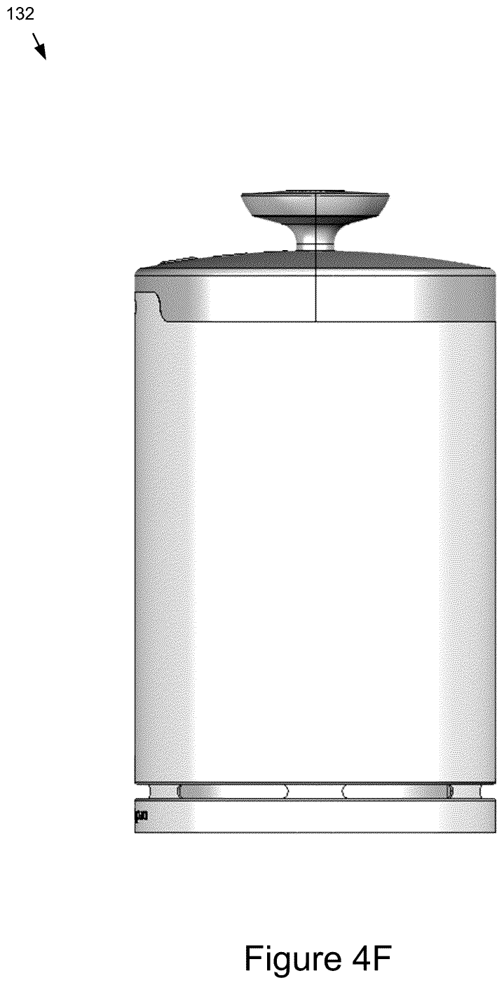

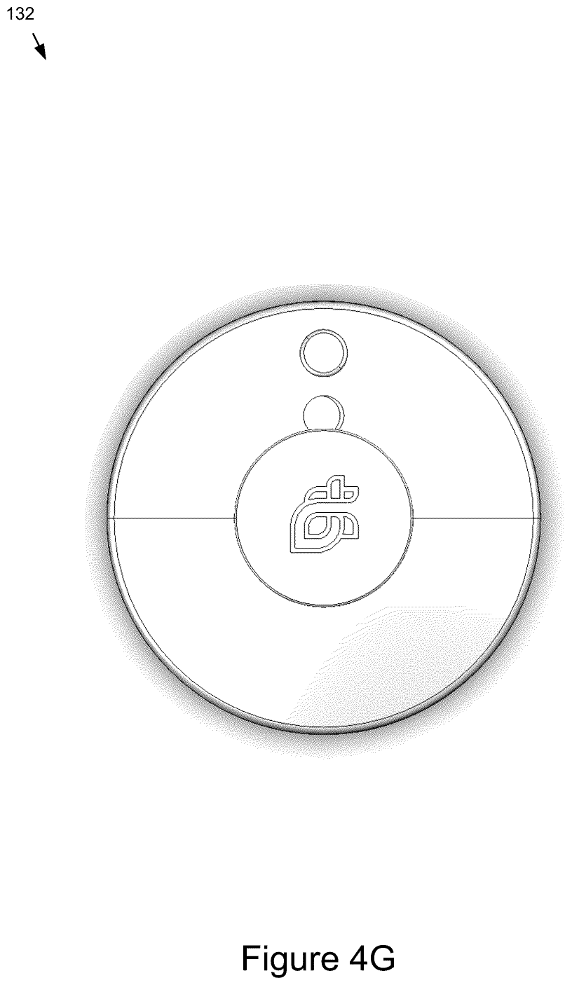

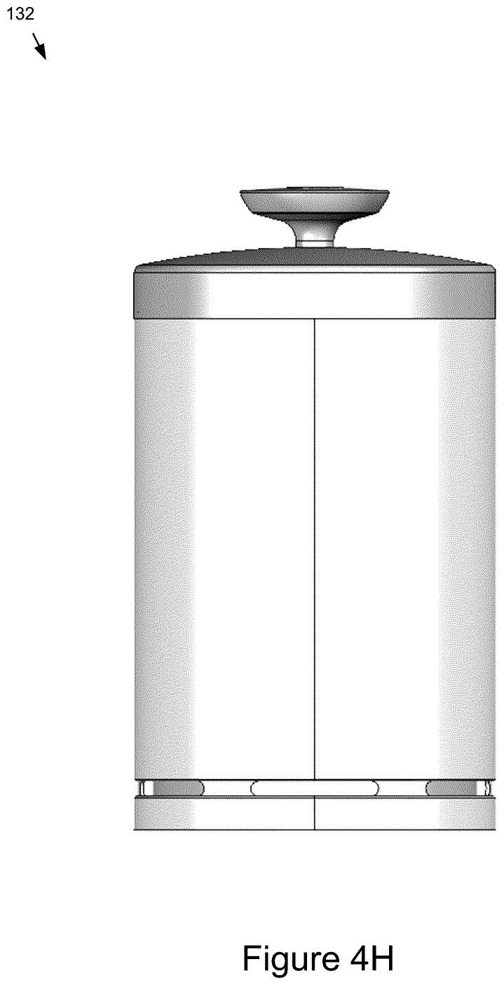

FIG. 4A is a top perspective view of an example scent dispensing device.

FIG. 4B is a bottom perspective view of the example scent dispensing device.

FIG. 4C is a front view of the example scent dispensing device.

FIG. 4D is a left side view of the example scent dispensing device.

FIG. 4E is a bottom view of the example scent dispensing device.

FIG. 4F is a right side view of the example scent dispensing device.

FIG. 4G is a top view of the example dispensing device.

FIG. 4H is a rear view of the example dispensing device.

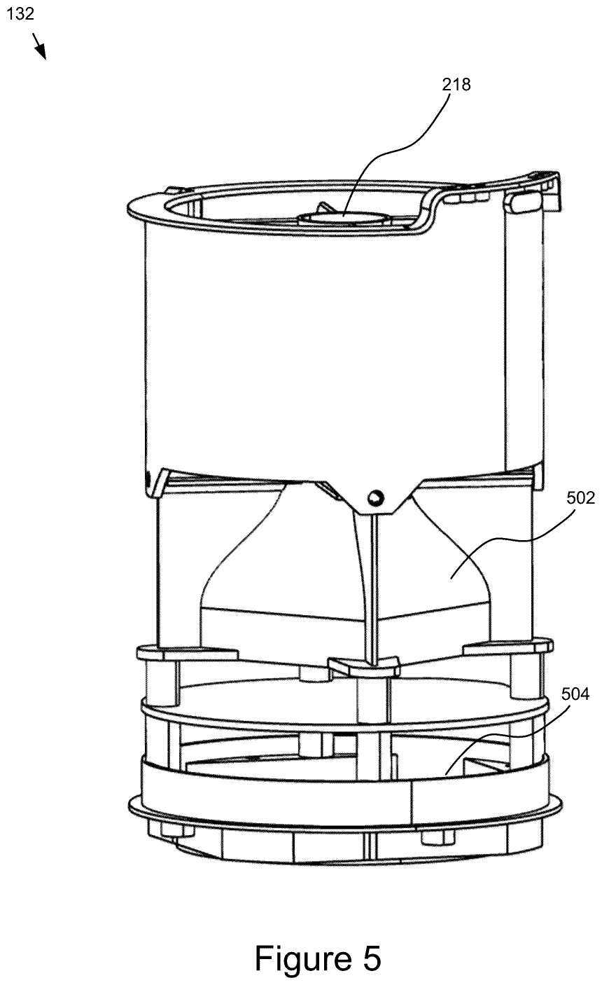

FIG. 5 is an example of the scent dispensing device 132 with the outer housing 200 (or housing/body) and top 204 removed. As can be seen in the example of FIG. 5, the scent dispensing device 132 includes a fan housing 502 which may hold a fan that draws air through the central airflow channel 218. The device also includes a space 504 to house other components for operation of the device, e.g., a battery, electronics, etc.

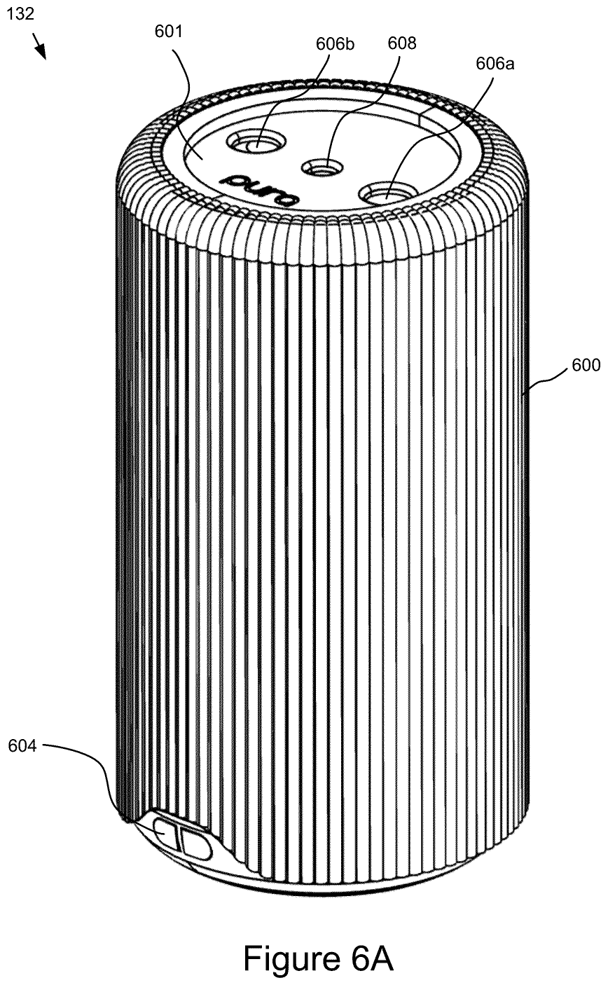

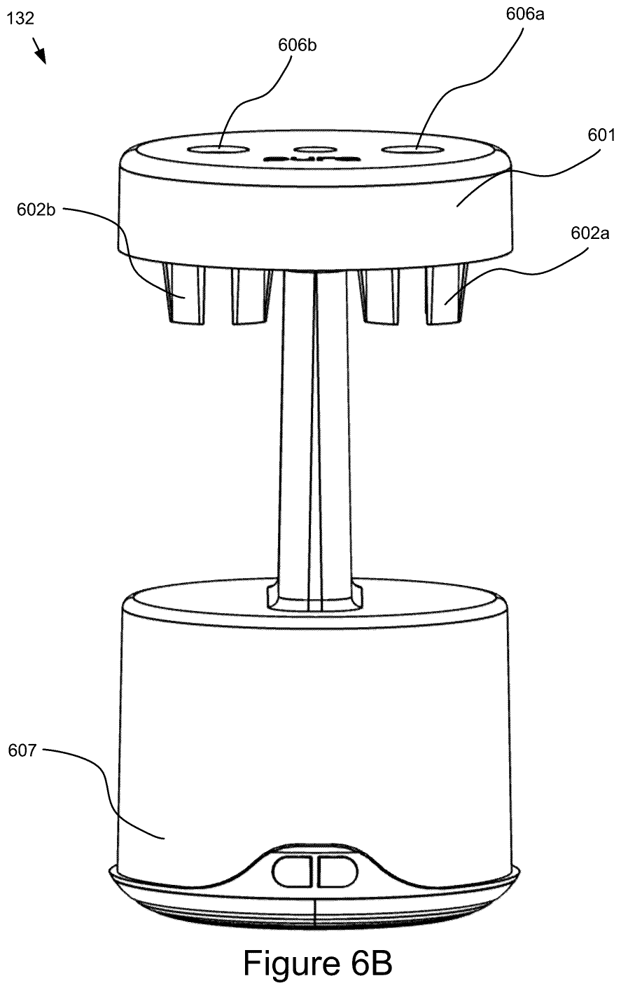

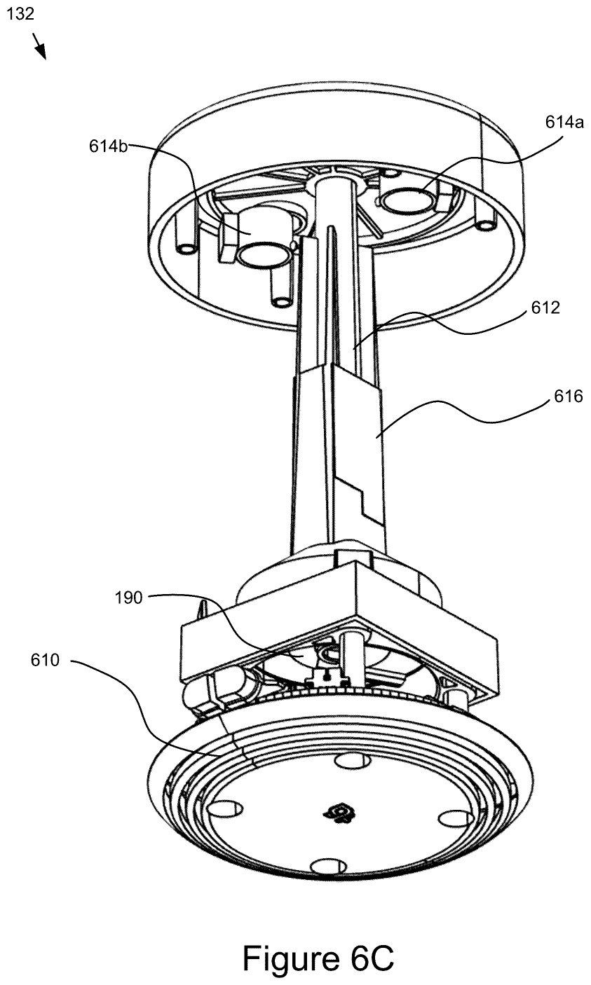

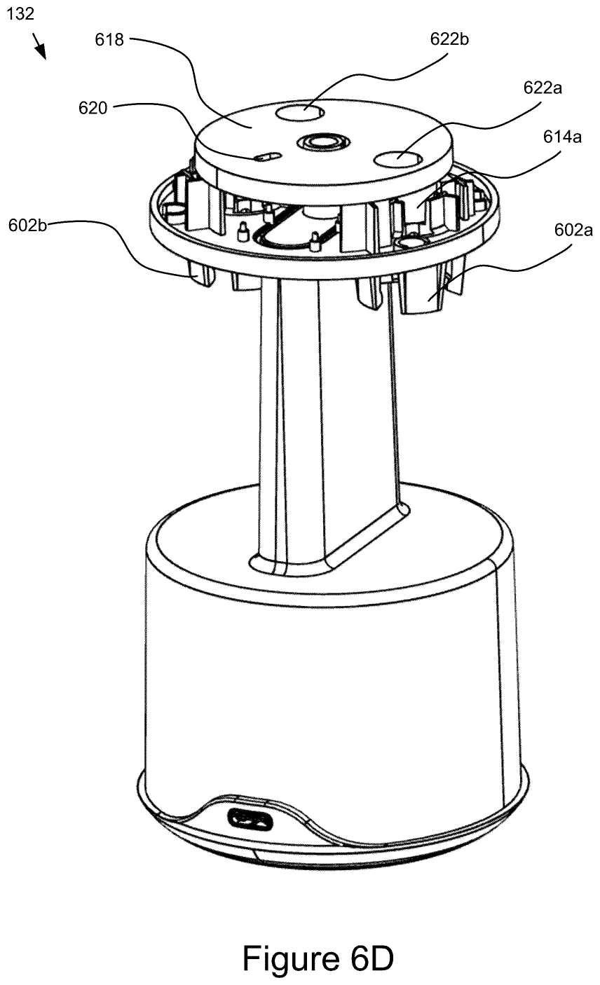

FIG. 6A is an example embodiment of another scent dispensing device 132. As shown in FIG. 6A, the scent dispensing device 132 may include an outer housing 600 that encloses the internals of the scent dispensing device 132, including the scent vial 250 and/or the fan 190. In some implementations, the outer housing 600 may have various types of decoration, such as the raised ridged shown in the example of FIG. 6A, or other types of images, prints, etc. in order for the outer housing 600 to mesh with other decorative styles on a countertop, table, room, etc. Similar to the other embodiments of a scent dispensing device 132 described herein, the example scent dispensing device 132 shown in FIG. 6A is configured to diffuse a scent through a top 601 of the scent dispensing device 132. As shown in the example of FIG. 6A, the top 601 may include one or more openings 606a and 606b through which scent may be diffused. In some embodiments, the openings 606a and 606b may be a hole or a mesh covered opening. In addition to openings 606a and 606b, the top 601 may include an opening 608 for air drawn through the device by fan 190 to exit the device and disperse the scent.

As shown, in some implementations, the scent dispensing device 132 may include one or more interactive areas 604, such as buttons, touch sensitive areas, dials, etc. with which a user may interact to change various settings or configurations on the scent dispensing device. For example, a user may interact with the one or more interactive areas 604, such as by pushing a button to increase/decrease a fan speed, or to activate a scent emission. In another example, a user may interact with the one or more interactive areas 604 to activate or turn off the scent dispensing device 132, etc.

In some implementations, the scent dispensing device 132 may have a wired connection to a power supply and be designed to rest on a counter or table while being plugged into a wall outlet via a power cord. In further implementations, the scent dispensing device 132 may be a battery powered device in order to allow for easy transport and movability.

FIG. 6B is another example of the scent dispensing device 132 introduced in FIG. 6A. In this example, the outer housing 600 has been removed to expose the inner components. As shown in this example, the scent dispensing device 132 may include two scent vials in some implementations. As shown, a scent vial (not shown) may be configured to attach to the scent vial holder 602a or 602b. Each scent vial holder 602a or 602b is configured to hold a scent vial below a corresponding opening 606a or 606b in the top 601 such that the scent emitted from the scent vial exits through the opening 606a or 606b. As shown in the example of FIG. 6B, the scent dispensing device 132 may include an inner housing 607 that covers the internal components of the scent dispensing device 132 (e.g., fan 190 and other electronic components).

FIG. 6C is another example of the scent dispensing device shown in FIG. 6A. As shown in FIG. 6C, the inner housing 607 has been removed to expose the internal components of the scent dispensing device 132. The internal components include fan 190, which is configured to draw air through openings 610 in the bottom of the scent dispensing device 132 and force the air upwards through the air channel 612 where it exits the scent dispensing device 132 through opening 608 (not shown in this view). As the air exits through opening 608, the airflow creates a pressure differential near openings 606a and 606b which draws scent molecules emitted from scent vials below the openings 606a and 606b into the air stream and disperses the scent molecules throughout the space. This method of distributing the scent molecules with the fan allows the scent to be broadly distributed without passing the scent (e.g., scented oil) over many other components of the scent dispensing device 132. Because the scent is forced outward based on the fan speed of the fan 190, the scent dispensing device 132 is able to fill a room or area with a scent faster as compared to conventional wall scent devices. In some implementations, the scent dispensing device 132 is able to fill a 1000 square foot to 1300 square foot area efficiently in a timeline that makes the scent appeal to the user (e.g., the scent diffuses before a user has come into a room and then left again, such as two to three minutes in some examples, or the scent diffusion does not have to be prefilled for a user to notice the scent diffusion, etc.). In some implementations, a user is able to set various schedules and settings for when and how large of an area the scent emission should be filling. In addition to distributing scent throughout the space, air pulled through the openings 610 and pushed through the scent dispensing device 132 can aid in cooling internal electronic components of the device.

In some embodiments, the scent dispensing device 132 includes heating elements 614a and 614b. The heating elements 614a and 614b may be configured to apply heat near a wick protruding from a scent vial 250. The heat encourages diffusion of the scent molecules from scent vial 250 such that an increased number of scent molecules exit through openings 606a and/or 606b to be dispersed by the airflow through opening 608.

Additionally, in some embodiments, scent dispensing device 132 may include a tag reader 616 to identify scent vials that are placed in the scent dispensing device. In some embodiments, the tag reader 616 may be a radio frequency identification (RFID) reader, a near field communication (NFC) device, an optical scanner, or the like, which can identify a scent vile having a passive identifier attached. Using the tag reader 616 to identify a scent vial can allow the scent dispensing device 132 to identify what scent is loaded in the device, how long the scent vial has been used, and other information about the scent vials. This information allows a user to better control the scent being dispensed from the scent dispensing device 132.

FIG. 6D is another example of the scent dispensing device shown in FIG. 6A. In FIG. 6D, the scent dispensing device 132 is shown with the outer housing 600 and the top 601 removed. In some embodiments, the scent dispensing device 132 includes a wick cover 618. The wick cover 618 may be configured to cover the wick of one or more scent vials (not shown) held in scent vial holder 602a or 602b. The scent dispensing device 132 may include an actuator (e.g., a motor, a servo assembly, or the like) (not shown) configured to interface with a slot 620 in the wick cover 618 and rotate the wick cover 618. The actuator may be configured to rotate the wick cover 618 such that the wick cover openings 622a and 622b are located at various positions. For example, the actuator may rotate the wick cover 618 to a first position where the wick cover opening 622a aligns with the wick of a scent vial held in scent vial holder 602a and opening 606a in top 601 while the wick of a scent vial 250 held in scent vial holder 602b is covered. This configuration isolates the scent being released from the scent dispensing device so there is less cross contamination of scents being dispersed. In some implementations, the actuator can rotate the wick cover 618 to various partially open settings (i.e., the wick cover opening 622a only partially aligns with opening 606a in top 601) to adjust the amount of scent being dispersed. In other embodiments, the actuator may be configured to rotate the wick cover 618 to a closed position (i.e., none of the wick cover openings 622a or 622b align with openings 606a or 606b in the top 601). This closed position can prevent scent being unintentionally dispersed into a space and also prevent evaporation of scent, leading scent vials to last longer. While the wick cover 618 and scent dispensing device 132 described in the examples of FIGS. 6A-6D include two scent vials and openings, it should be understood that any number of scent vials and openings are possible.

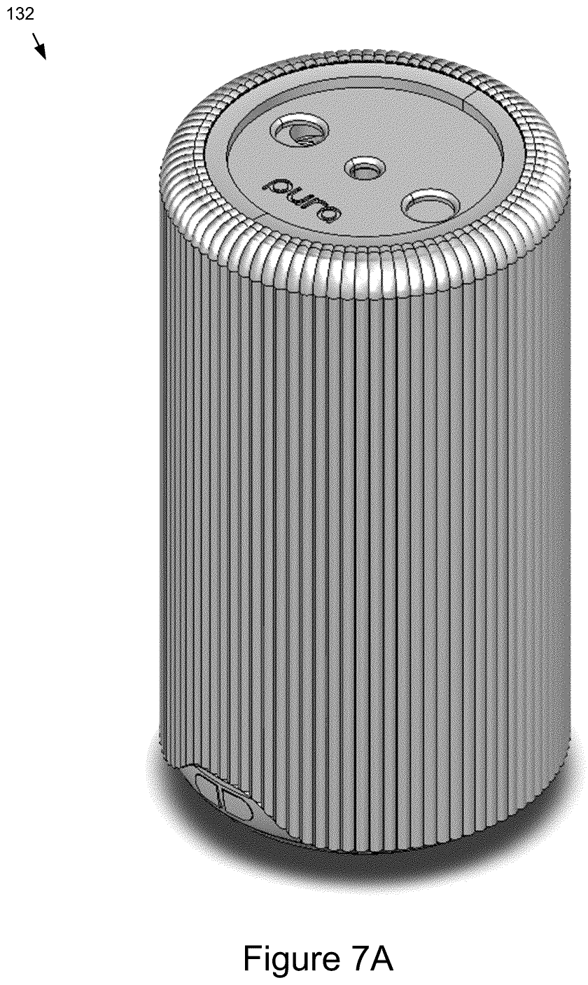

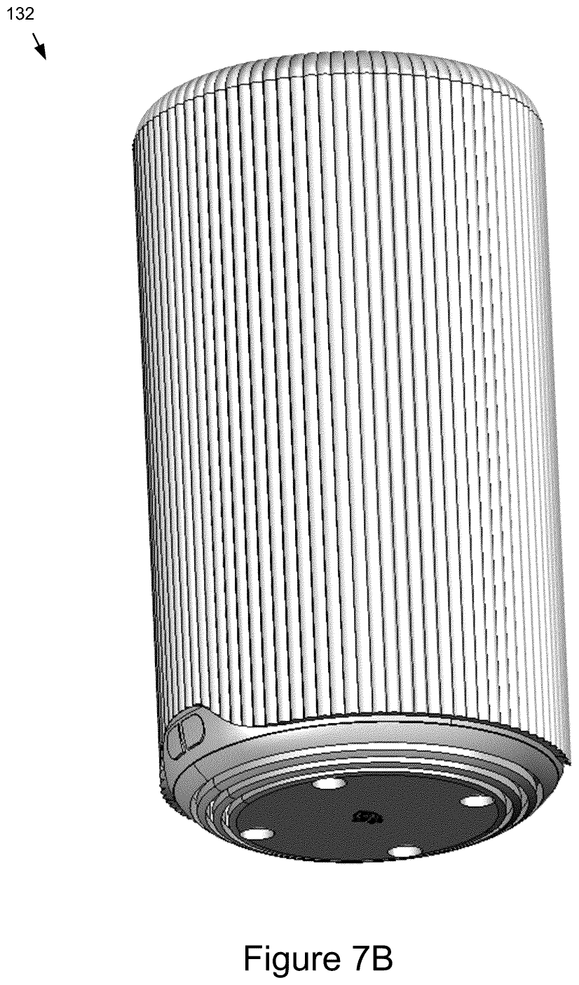

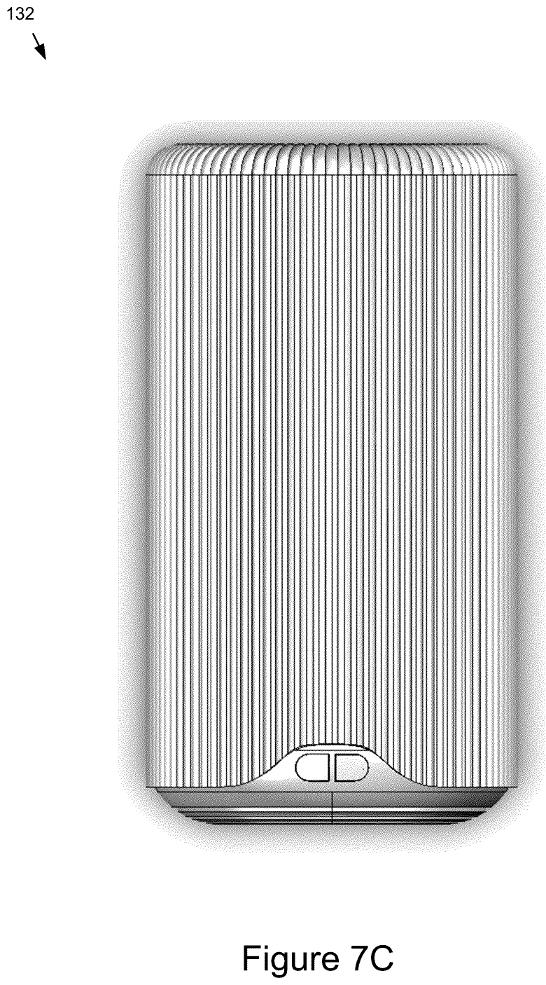

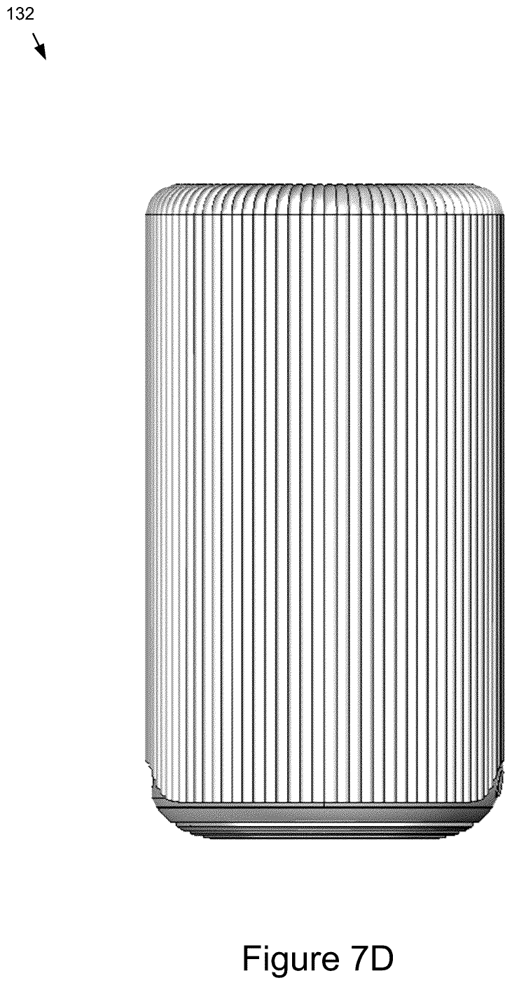

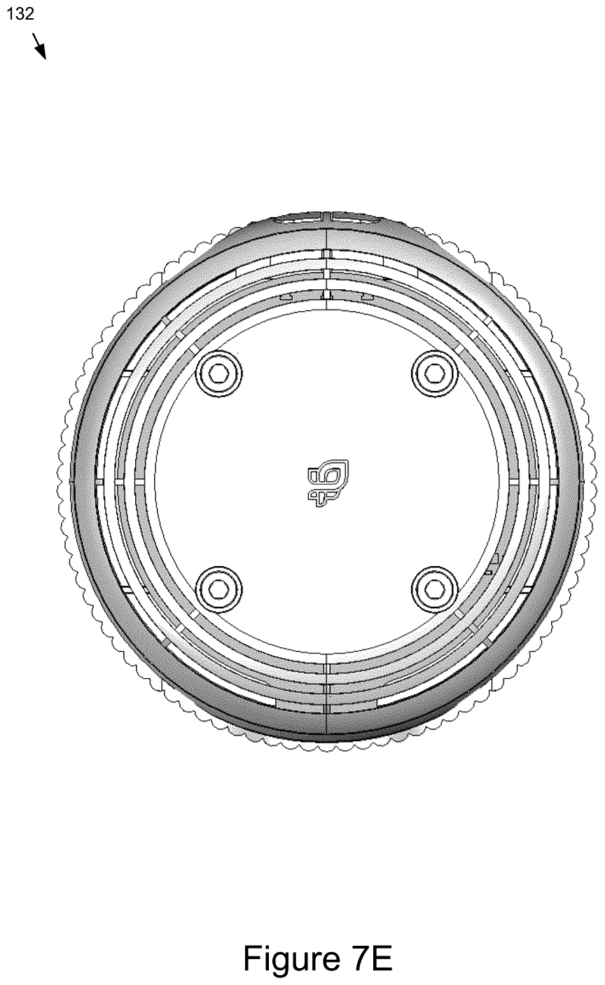

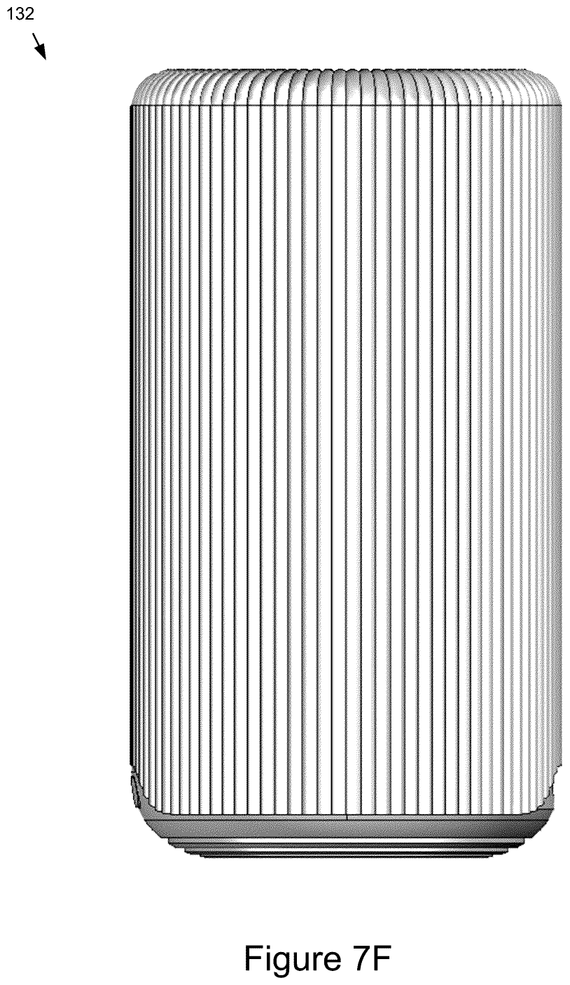

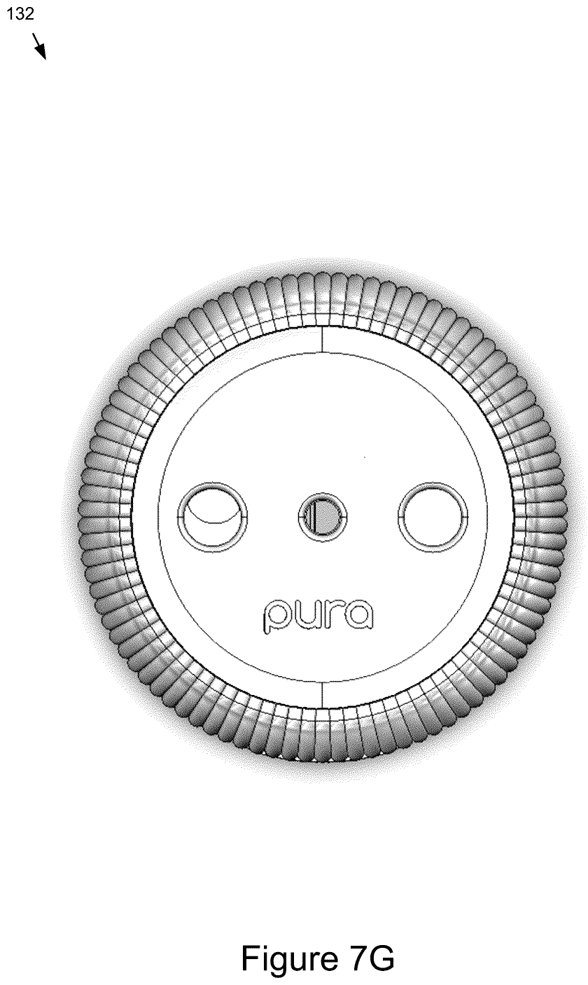

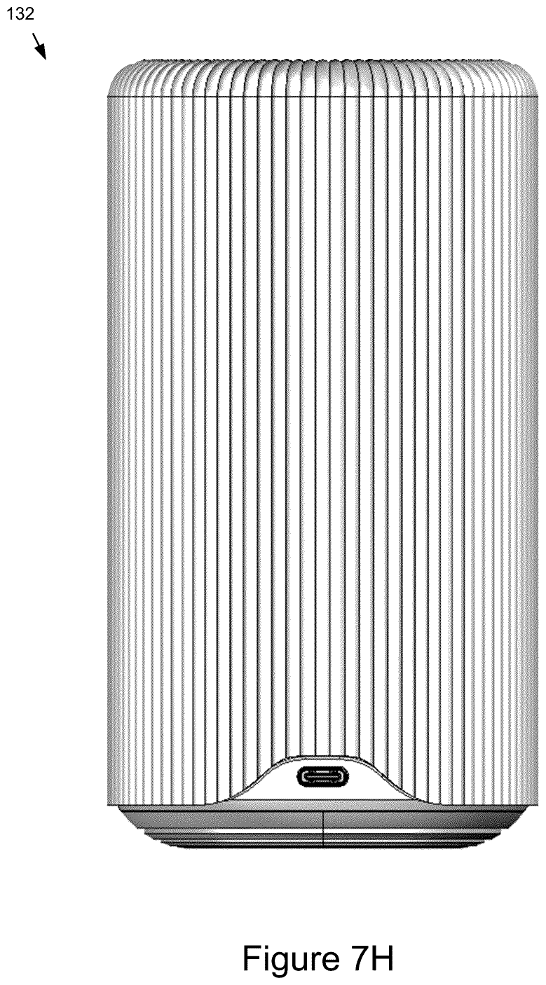

FIG. 7A is a top perspective view of another example scent dispensing device.

FIG. 7B is a bottom perspective view of the example scent dispensing device.

FIG. 7C is a front view of the example scent dispensing device.

FIG. 7D is a left side view of the example scent dispensing device.

FIG. 7E is a bottom view of the example scent dispensing device.

FIG. 7F is a right side view of the example scent dispensing device.

FIG. 7G is a top view of the example dispensing device.

FIG. 7H is a rear view of the example dispensing device.

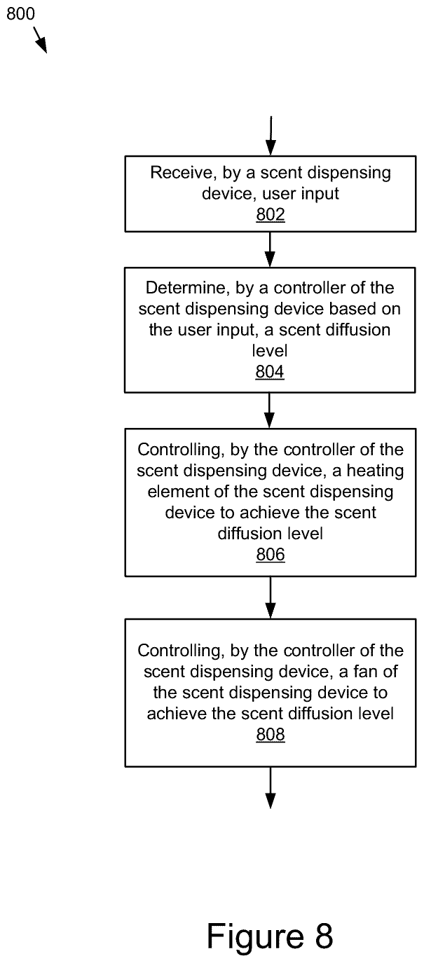

FIG. 8 is a flowchart of an example method 800 for dispensing a scent using a scent dispensing device 132. At 802, the scent dispensing device 132 receives user input including scent parameters. The scent parameters may be received from a scent application running on user device 106 or the dispenser management application 160 running on the dispenser management server. In some embodiments the scent parameters are received via network 102 and in other embodiments the scent parameters are received directly from the mobile device via a direct wired or wireless connection. The scent parameters may include, for example, scent selection, run time, scent mixture, room size, time of day, ambient temperature, scent level preference, etc.

At 804, the scent dispensing device 132 determines a diffusion level using the scent parameters. The diffusion level is a setting of the device to control the heater and fan in the scent dispensing device 132 which determines the amount of scent produced by the scent dispensing device 132. By determining a diffusion level of the scent, the scent dispensing device 132 can preserve the life of the scent vial and provide customized scent levels based on user preferences and environmental factors, e.g., causing the fan to blow more in a larger space or less in a smaller space and to apply the proper amount of heat to the scent vial 250 to achieve optimal scent levels. Essentially, the scent dispensing device 132 can create a stable and consistent scenting experience for the user 112 using the scent parameters to determine a scent diffusion level.

In some implementations, the diffusion level may be also based on a user setting, such as a low level, medium level, or high level that may be input by the user 112 to set a scent preference. In some implementations, the user setting can be changed by providing input on the user device 106, while in further implementations an input device may be used, such as a button or slider, to adjust the user setting. In some implementations, the user settings may also include various modes, such as burst modes, wind down modes, etc. and the diffusion level can be further changed based on the selected modes. In some implementations, the output device 192, such as a row of lights, etc., may signal to the user 112 the selected settings and diffusion level.

At 806 and 808, using the determined diffusion level, the scent dispensing device 132 causes the heating elements and/or fan to operate and force air through the device and out of the top over the scent openings to disperse the scent into the space. Using the fan, the scent can be quickly dispersed into the space and is more noticeable to the user 112. In some implementations, the scent dispensing device 132 can operate the fan on various pulses in order to reduce the effect of nose blindness, allowing the scent to diffuse and then settle while the fan is off for a period of time before another pulse of the fan emits another burst of scent for a period of time.

In some implementations, the scent dispensing device 132 can automatically stop diffusing scent (e.g., in response to a schedule or presence sensors, etc.). By stopping diffusing scent, the lifetime of the scent is increased because it is not being dispersed while not in use and the user 112 does not have to manage operation of the scent dispensing device 132 manually. For example, in some implementations, when the scent dispensing device 132 connects to the user device 106, such as by a Bluetooth connection or detects a connection within a geofence, the scent dispensing device 132 may begin scent dispensation and similarly stop operation when a connection with the user device 106 is no longer detected. In some implementations, the scent dispensing device 132 can specifically look for the Bluetooth beacon from the user device 106 to indicate when to operate or stop operation.

The foregoing description, for purpose of explanation, has been described with reference to various embodiments and examples. However, the illustrative discussions above are not intended to be exhaustive or to limit the invention to the precise forms disclosed. Many modifications and variations are possible in view of the above teachings. The various embodiments and examples were chosen and described in order to best explain the principles of the invention and its practical applications, to thereby enable others skilled in the art to utilize the innovative technology with various modifications as may be suited to the particular use contemplated. For instance, it should be understood that the technology described herein can be practiced without these specific details in some cases. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and symbolic representations of operations on data bits within a computer memory. An algorithm is here, and generally, conceived to be a self-consistent set of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms such as “processing,” “computing,” “calculating,” “determining,” “displaying,” or the like, refer to the action and methods of a computer system that manipulates and transforms data represented as physical (electronic) quantities within the computer system's registers and memories into other data similarly represented as physical quantities within the computer system memories or registers or other such information storage, transmission or display devices.

A data processing system suitable for storing and/or executing program code, such as the computing system and/or devices discussed herein, may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input or I/O devices can be coupled to the system either directly or through intervening I/O controllers. The data processing system may include an apparatus may be specially constructed for the required purposes, or it may comprise a general-purpose computer selectively activated or reconfigured by a computer program stored in the computer.

The foregoing description has been presented for the purposes of illustration and description. It is not intended to be exhaustive or to limit the specification to the precise form disclosed. Many modifications and variations are possible in light of the above teaching. It is intended that the scope of the disclosure be limited not by this detailed description, but rather by the claims of this application. As will be understood by those familiar with the art, the specification may be embodied in other specific forms without departing from the spirit or essential characteristics thereof. Likewise, the particular naming and division of the modules, routines, features, attributes, methodologies and other aspects may not be mandatory or significant, and the mechanisms that implement the specification or its features may have different names, divisions, and/or formats.

Furthermore, the modules, routines, features, attributes, methodologies and other aspects of the disclosure can be implemented as software, hardware, firmware, or any combination of the foregoing. The technology can also take the form of a computer program product accessible from a computer-usable or computer-readable medium providing program code for use by or in connection with a computer or any instruction execution system. Wherever a component, an example of which is a module or engine, of the specification is implemented as software, the component can be implemented as a standalone program, as part of a larger program, as a plurality of separate programs, as a statically or dynamically linked library, as a kernel loadable module, as firmware, as resident software, as microcode, as a device driver, and/or in every and any other way known now or in the future. Additionally, the disclosure is in no way limited to implementation in any specific programming language, or for any specific operating system or environment. Accordingly, the disclosure is intended to be illustrative, but not limiting, of the scope of the subject matter set forth in the following claims.

What is claimed is:

1. A scent dispensing device, comprising:
a scent vial holder configured to hold a scent vial;
a top having a first plurality of openings, the first plurality of openings including a scent opening and an airflow opening;
a heater configured to apply heat to the scent vial; and
a fan configured to cause a flow of air to pass through the airflow opening creating a difference in air pressure between the airflow opening and the scent opening such that scent from the scent vial is drawn through the scent opening and into the flow of air.

2. The scent dispensing device of claim 1, further comprising:
a controller configured to control the heater and the fan to achieve a selected scent diffusion level based on user input.

3. The scent dispensing device of claim 2, further comprising a wick cover having a second plurality of openings, the wick cover configured to at least partially close the scent opening.

4. The scent dispensing device of claim 3, further comprising an actuator coupled with the wick cover, the actuator configured to adjust a position of the wick cover.

5. The scent dispensing device of claim 4, wherein the controller is further configured to control the actuator such that the second plurality of openings of the wick cover are positioned to further achieve the selected scent diffusion level.

6. The scent dispensing device of claim 1, further comprising a tag reader configured to read a tag coupled with the scent vial.

7. The scent dispensing device of claim 1, further comprising:
a base having a third plurality of openings; and
an air channel positioned between the fan and the airflow opening, wherein the fan is configured to draw air through the third plurality of openings and through the air channel such that scent molecules are not drawn over internal components of the scent dispensing device.

8. A scent dispensing device, comprising:
a scent vial holder configured to hold a plurality of scent vials;
a tag reader configured to read tags coupled with the plurality of scent vials;
a top having a first plurality of openings, the first plurality of openings including a plurality of scent openings and an airflow opening;
a plurality of heating elements configured to selectively apply heat to the plurality of scent vials;
a fan configured to cause a flow of air to pass through the airflow opening creating a difference in air pressure between the airflow opening and the plurality of scent openings such that one or more scents from one or more of the plurality of scent vials is drawn through the plurality of scent openings and into the flow of air; and
a controller configured to control the plurality of heating elements and the fan to achieve a selected scent diffusion level of the one or more scents.

9. The scent dispensing device of claim 8, further comprising a wick cover having a second plurality of openings, the wick cover configured to at least partially close one or more of the plurality of scent openings.

10. The scent dispensing device of claim 9, further comprising an actuator coupled with the wick cover, the actuator configured to adjust a position of the wick cover.

11. The scent dispensing device of claim 10, wherein the controller is further configured to control the actuator such that the second plurality of openings of the wick cover are positioned to further achieve the selected scent diffusion level.

12. The scent dispensing device of claim 11, wherein the controller is further configured to identify a scent contained in each of the plurality of scent vials based on an output from the tag reader.

13. The scent dispensing device of claim 12, wherein the controller is further configured to:
selectively apply heat to a first scent vial of the plurality of scent vials; and
control the actuator such that the wick cover is not covering a first scent opening corresponding to the first scent vial and is covering remaining scent openings of the plurality of scent openings to achieve the selected scent diffusion level.

14. The scent dispensing device of claim 8, further comprising:
a base having a third plurality of openings; and
an air channel positioned between the fan and the airflow opening, wherein the fan is configured to draw air through the third plurality of openings and through the air channel such that scent molecules are not drawn over internal components of the scent dispensing device.

15. A method comprising:
receiving, by a controller of a scent dispensing device, user input;
determining, by the controller of the scent dispensing device based on the user input, a scent diffusion level;
controlling, by the controller of the scent dispensing device, a heating element of the scent dispensing device to achieve the scent diffusion level; and
controlling, by the controller of the scent dispensing device, a fan of the scent dispensing device to achieve the scent diffusion level.

16. The method of claim 15, wherein determining the scent diffusion level comprises identifying a plurality of scent parameters and determining the scent diffusion level based on the plurality of scent parameters.

17. The method of claim 16, wherein the plurality of scent parameters include one or more of: a scent selection, a run time, a scent mixture, a room size, a time of day, an ambient temperature, and a scent level preference.

18. The method of claim 15, wherein controlling the fan of the scent dispensing device further comprises operating the fan in a pulse mode such that bursts of scent are dispersed to achieve the scent diffusion level.

19. The method of claim 15, further comprising:
controlling, by the controller of the scent dispensing device, an actuator coupled with a wick cover of the scent dispensing device to cover a scent vial wick when the scent dispensing device is not in use.

20. The method of claim 15, further comprising:
identifying, by the controller of the scent dispensing device, a scent vial based on input from a tag reader configured to read a tag coupled with the scent vial held in the scent dispensing device.
